(19) United States
(12) Patent Application Publication
(10) Pub. No.: US 2015/0330003 A1
(43) Pub. Date: Nov. 19, 2015

(54) PATTERNED NONWOVEN AND METHOD OF MAKING THE SAME USING A THROUGH-AIR DRYING PROCESS

(71) Applicant: First Quality Nonwovens, Inc., Great Neck,, NY (US)

(72) Inventor: W. Andrew Coslett, Drums, PA (US)

(21) Appl. No.: 14/711,393

(22) Filed: May 13, 2015

Related U.S. Application Data
(60) Provisional application No. 61/992,731, filed on May 13, 2014.

(51) Int. Cl. D04H 3/00; A61F 13/53

(52) U.S. Cl. CPC D04H 3/00; A61F 13/53; A61F 2013/15406

(57) ABSTRACT

A nonwoven web including a first layer that includes continuous fibers having a first polymer component and a second layer that includes continuous fibers having a second polymer component. The melting point of the second polymer component is less than the melting point of the first polymer component, and the first layer is hydroentangled with the second layer.

### RELATED APPLICATION

This application is a non-provisional based on and claiming priority to U.S. Provisional Application No. 61/992,731, filed May 13, 2014, the contents of which are incorporated herein by reference in their entirety.

### FIELD

The present disclosure relates to systems and methods for producing nonwoven webs, and in particular to systems and methods for producing patterned nonwoven webs.

### BACKGROUND

A conventional option for producing high loft, resilient patterned nonwovens is through the use of a carded, through air bonded structure which is then subsequently embossed to provide a visually distinct structure. The use of carded technology allows the selection of crimped sheath core bicomponent fibers or side by side bicomponent fibers where the core, combined with the crimp provides stiffness and resilience while the sheath material provides lower melting and bonding for structural integrity.

It is well known that spun melt nonwoven fabrics offer the potential for lower cost manufacturing due to two main factors. The first is the typically higher line speeds that the spunmelt processes achieve while the second is the generation of the nonwoven fiber directly from the resin thus eliminating the cost of staple fiber production. However, it has been difficult to produce patterned, high loft, spunmelt nonwovens without the use of complicated processes.

### SUMMARY OF THE INVENTION

An object of this invention is to increase resilience and loft of a patterned spunmelt fabric produced by hydroentangling through the use of a combination of polymer components that have different melting points.

The present invention is directed to a system and method for making a patterned nonwoven fabric in which a nonwoven web is formed and then subjected to a thermal bonding step, a hydroentanglement step and a through-air drying (TAD) process. The through-air drying process includes a first TAD step that dries the web and a second TAD step that melts and/or softens a low melting component of the web.

An advantage provided by the present invention is that the higher melting fibers can be used as a protective layer for the belts and for the drying drum. The higher melting component is laid down first on the spin belt and the lower melting fibers are a second component. The higher melt component minimizes the likelihood that the lower melting fibers will stick to the web. In addition, even though the layers are intermingled through the hydroentangling process, the higher melt components are the predominant components in direct contact with the dryer drum surface. This structure helps to minimize the risk of the lower melt component sticking to the drum.

A nonwoven web according to an exemplary embodiment of the present invention comprises: a first layer comprising continuous fibers including a first polymer component; and a second layer comprising continuous fibers including a second polymer component, the melting point of the second polymer component being less than the melting point of the first polymer component, the first layer being hydroentangled with the second layer.

In an exemplary embodiment, the first polymer component is polypropylene.

In an exemplary embodiment, the second polymer component is polyethylene.

In an exemplary embodiment, the continuous fibers of the second layer further include the first polymer component.

In an exemplary embodiment, the continuous fibers of the second layer are bicomponent fibers.

In an exemplary embodiment, the first layer comprises a pattern.

In an exemplary embodiment, the bicomponent fibers are arranged in a side by side configuration.

In an exemplary embodiment, the nonwoven web has a basis weight within the range of 30 gsm to 35 gsm and a tensile strength in the machine direction of at least 1400 g/cm.

In an exemplary embodiment, the nonwoven web has a tensile strength in the cross direction of at least 500 g/cm.

In an exemplary embodiment, the nonwoven web has a percent elongation in the machine direction within the range of 50 to 100.

In an exemplary embodiment, an absorbent article includes from top to bottom a topsheet, an absorbent core, and a backsheet, wherein the topsheet comprises a nonwoven web that comprises: a first layer comprising continuous fibers including a first polymer component; and a second layer comprising continuous fibers including a second polymer component, the melting point of the second polymer component being less than the melting point of the first polymer component, the first layer being hydroentangled with the second layer.

In an exemplary embodiment, the nonwoven web has a basis weight within the range of 60 gsm to 70 gsm and a tensile strength in the machine direction of at least 3400 g/cm.

In an exemplary embodiment, the nonwoven web has a tensile strength in the cross direction of at least 1100 g/cm.

In an exemplary embodiment, the nonwoven web has a percent elongation in the machine direction within the range of 60 to 130.

According to an exemplary embodiment of the present invention, a method of making a patterned nonwoven web comprises: forming a nonwoven web comprising: a first layer comprising continuous fibers; and a second layer comprising continuous fibers, the melting point of the continuous fibers of the second layer being less than the melting point of the continuous fibers of the first layer; thermal bonding the first layer to the second layer; subjecting the thermally bonded first and second layers to a hydroentanglement process so as to form a pattern in the first layer; and through air drying the hydroentangled first and second layers so that the continuous fibers of the second layer melt.

In an exemplary embodiment, the step of forming a nonwoven web comprises a spunmelt process.

In an exemplary embodiment, the thermal bonding is done at a reduced temperature relative to a standard temperature for thermal bonding.

In an exemplary embodiment, the continuous fibers of the first layer are made of polypropylene.

In an exemplary embodiment, the continuous fibers of the second layer are made of polyethylene.

In an exemplary embodiment, the through-air drying step comprises a two-stage through-air drying process.

In an exemplary embodiment, the two-stage through-air drying process comprises a first stage through-air drying process in which the nonwoven web is dried and a second stage through-air drying process in which the continuous fibers of the second layer are melted.

Other features and advantages of embodiments of the invention will become readily apparent from the following detailed description, the accompanying drawings and the appended claims.

### BRIEF DESCRIPTION OF THE DRAWINGS

The features and advantages of exemplary embodiments of the present invention will be more fully understood with reference to the following, detailed description when taken in conjunction with the accompanying figures, wherein:

FIG. 1 is a representative diagram of a system for forming a patterned nonwoven web according to an exemplary embodiment of the present invention;

FIG. 2 is a cross-sectional view of a nonwoven web according to an exemplary embodiment of the present invention; and

FIG. 3 is a chart showing dryer temperature vs. abrasion rating of nonwoven webs according to exemplary embodiments of the present invention.

### DETAILED DESCRIPTION

The present invention is directed to a method of making a patterned nonwoven web using hydroentanglement. According to an exemplary embodiment, a spunbond and/or SMS nonwoven web is provided having at least one layer having a low melt component. The lower melting component should have a melting point at least 20 degrees C. lower than the main polymer component of the web and should make up at least 20% of the web. The web may be thermally bonded, and then subjected to hydroentanglement. After hydroentanglement, the web is subjected to a through-air drying (TAD) process. The TAD process includes two TAD steps, including a first TAD step that dries the web and a second TAD step that melts and/or softens the low melting component of the web. The low melting component provides additional bonding to the web so that the pattern is more fixed and holds it appearance under compression. It should be appreciated that one or two through-air dyers may be used to perform the two TAD steps.

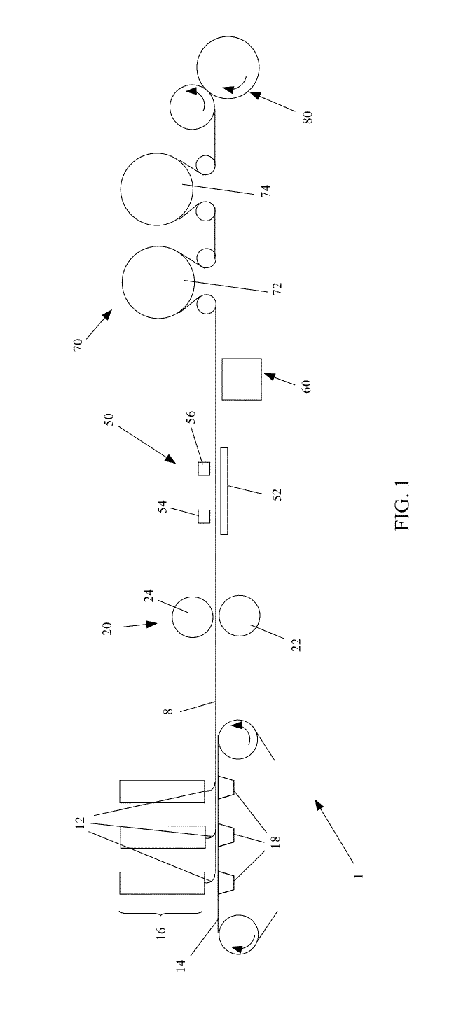

FIG. 1 is a representative diagram of a system, generally designated by reference numeral 1, for forming a patterned nonwoven web according to an exemplary embodiment of the present invention. The method begins with a nonwoven web of continuous fibers formed by processes well known in the art. Preferably the web is a “meltspun”—that is, a meltblown, spunbond or combination thereof. In a preferred embodiment, the fibers are thermoplastic or spinnable polymers selected from the group consisting of polyolefins, polyesters, polyamides, copolymers thereof (with olefins, esters, amides or other monomers) and blends thereof. As used herein the term “blend” includes either a homogeneous mixture of at least two polymers or a non-homogeneous mixture of at least two physically distinct polymers such as bicomponent fibers. Preferably the fibers are polyolefins selected from the group consisting of polyethylene, polypropylene, propylene-butylene copolymers thereof and blends thereof, including, for example, ethylene/propylene copolymers and polyethylene/polypropylene blends. According to an exemplary embodiment, the fibers are a mix of polypropylene and polyethylene (low melt component) bicomponent fibers in a side by side configuration having a linear mass density of 1.8 denier. Fibers having linear mass densities greater than or less than 1.8 denier may be used.

As shown in FIG. 1, a spunmelt nonwoven web 8 according to an exemplary embodiment of the invention is made of continuous fibers 12 that are laid down on a moving conveyor belt 14 in a randomized distribution. In a typical spunmelt process, resin pellets are processed under heat into a melt and then fed through a spinneret to create hundreds of fibers 12 by use of a drawing device 16. Multiple spinnerets or beams may be used to provide an increased density of spunbond fibers. Although FIG. 1 shows the use of three beams, any number of beams may be used, such as two or four beams.

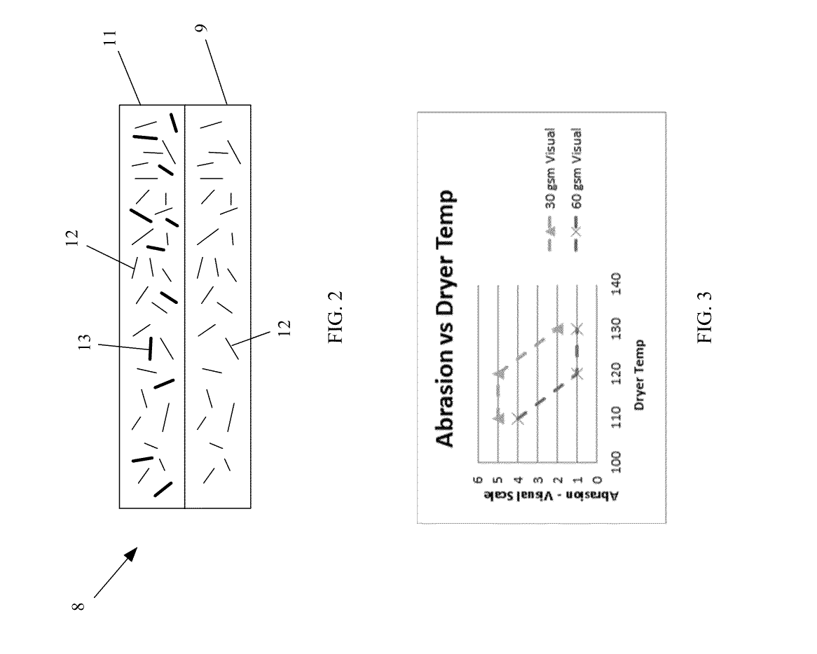

As shown in FIG. 2, according to an exemplary embodiment of the invention, the nonwoven web 8 includes a two-layer structure, including a bottom layer 9 and a top layer 11. The top layer 11 includes at least some fibers with a lower melt component 13, such as, for example, polyethylene fibers. The lower melt fibers 13 have a lower melting point than the fibers 12, which form the main component fibers of the top and bottom layers 9, 11. In an alternative embodiment, the bottom layer 9 may also include at least some low melt fibers 13.

Jets of a fluid (such as air) cause the fibers 12, 13 to be elongated, and the fibers 12, 13 are then blown or carried onto a moving web 14 where they are laid down and sucked against the web 14 by suction boxes 18 in a random pattern to create a fabric structure.

The web 8 then passes through a thermal bonding station 20 where the nonwoven web 8 is lightly thermally bonded. In order to achieve the light bonding, the thermal bonding process is typically set 20-40 degrees C. cooler than normal for a particular polymer type. For example, a normal calender bonding temperature for a PP spunbond web would be approximately 160 degrees C., so that light bonding is achieved using temperatures in the range of 125-140 degrees C. The thermal bonding process pre-bonds the continuous fibers 12 of the nonwoven web 8. The pre-bonds maintain the integrity of the nonwoven web 8 while the web is conveyed down-line to other processing stations, but are not necessarily intended to remain in the web as part of the final product. The bond area of the nonwoven web 10 is in the range of 10% to 25%.

The typical thermal bonding station 20 includes a calender 22 having a bonding roll 24 defining a series of identical raised points or protrusions (not shown). The bonding points are generally equidistant from each other and are in a uniform and symmetrical pattern extending in all directions (that is, an isotropic pattern), and therefore in both the machine direction (MD) and the cross direction (CD). Alternatively, the thermal bonding station 20 may have an ultrasonic device or a through-air bonding device using air at elevated temperatures sufficient to cause thermal bonding.

It is also possible that the two layered structure is composed of filament types that are thermally incompatible and thus cannot be effectively bonded. One means to address this issue is to spray a curtain of polyolefinic hot melt adhesive in the form of fine filaments onto the structure. Such filaments can be applied in either a continuous or discontinuous manner to co-join the thermally incompatible layers. This would enable the combined web structure to be conveyed to downstream processing steps without undue distortion or necking.

After thermal bonding, the nonwoven web 8 proceeds to a hydroentanglement station 50. At the hydroentanglement station 50, the nonwoven web 8 is subjected to hydroentanglement while proceeding over a foraminous forming surface 52, the movement of which is synchronized with the movement of the nonwoven web 8. In an exemplary embodiment, the forming surface 52 is a perforated support screen having an open area within the range of 35% to 75%, and in an exemplary embodiment has an open area of 50%. The second hydroentanglement step involves the use of a first injector 54 and a second injector 56. The first injector 54 includes a jet strip having one or more rows of 120 micron holes spaced 0.6 mm apart that eject water at a pressure within a range of 180 bar to 300 bar, and in an exemplary embodiment eject water at a pressure of 250 bar. The second injector 56 includes a jet strip having a single row of 120 micron holes spaced 0.6 mm apart that eject water at a pressure within a range of 180 bar to 300 bar, and in an exemplary embodiment eject water at a pressure of 200-250 bar. The hydroentanglement process is intended to intermingle the fibers in the nonwoven web 8 and to cause the nonwoven web 8 to assume the inverse pattern of the two-dimensional forming surface 52. The bottom layer 9 of the nonwoven web 8 is in contact with the foraminous forming surface 52 during the hydroentanglement process.

In exemplary embodiments, the nonwoven web 8 may be subjected to a two-step hydroentanglement process, where the first hydroentanglement step is intended to break the thermal bonds and intermingle the fibers in the nonwoven web 8 and the second hydroentanglement step is intended to further intermingle the fibers in the nonwoven web 8. The first hydroentanglement step may take place while the nonwoven web 8 travels over a foraminous forming drum.

After hydroentanglement, the nonwoven web 8 is subjected to dewatering at dewatering station 60. Dewatering station 60 may include suction boxes that draw water out of the nonwoven web 8 as the nonwoven web 8 progresses over a dewatering wire or dewatering drum.

After dewatering, the nonwoven web 8 may be treated with various chemicals, such as, for example, surfactants, at a treatment station (not shown). The treatment station may include one or more kiss rolls that apply the chemicals to the nonwoven web 8.

The nonwoven web 8 is then brought to a drying station 70 for drying before the finished material is reeled up at winding station 80 and converted. Within the drying station 70, the nonwoven web 8 may be dried using conventional through air drying processes. For example, the drying station 70 may include a through air drier manufactured by Andritz-Perfojet of Montbonnot, France. In an exemplary embodiment of the invention, two through air drying stages 72, 74 are used in series. The first stage 72 dries the web 8 and the second stage 74 serves to melt and/or soften the low melting temperature fibers 13 within the top layer 11. Melting and/or softening of the fibers 13 provides additional bonding to the web 8 so that the pattern is more fixed and holds its appearance under compression.

Before conversion, the nonwoven web 8 may be subjected to treatments, such as corona or plasma treatment, treatment with chemicals of any desired kind, etc. Corona or plasma treatment is preferably made after drying while chemicals may be added either to the fiber dispersion or after dewatering of the web by spraying, printing or the like.

The nonwoven web 8 may be incorporated into a nonwoven laminate. The nonwoven laminate may include additional layers of continuous fibers such as spunbond fibers and meltblown fibers and may include composite nonwovens such as spunbond-meltblown-spunbond laminates. The nonwoven laminate may also include short fibers such as staple fibers or may include pulp fibers. The nonwoven laminate may also include superabsorbent material, either in particulate form or in a fiberized form. The laminate may be formed through conventional means, including but not limited to thermal bonding, ultrasonic bonding, chemical bonding, adhesive bonding and/or hydroentanglement.

The nonwoven web 8 may be incorporated into various absorbent articles, such as but not limited to, diapers, training pants, adult protective underwear, bladder control pads, feminine hygiene pads, tampons, and changing pads. In an exemplary embodiment, the nonwoven web 8 may be used as a topsheet in an absorbent article having a topsheet, absorbent core and backsheet. In another embodiment, the nonwoven web 8 may be located between the topsheet and absorbent core of an absorbent article. In this embodiment, the nonwoven web 8 can function as a surge layer or acquisition/distribution layer.

The following Examples illustrate various objects and advantages of the present invention:

### EXAMPLE 1

A three layer patterned non-woven web was provided, with the bottom layer being 100% polypropylene and the top two layers being made of 60/40 polypropylene/polyethylene side-by-side bicomponent fibers. The web had a basis weight of 30.0 gsm and a thickness of 0.363 mm. The patterned non-woven web was produced by first bonding the layers together and subjecting the web to hydroentanglement and then a two-stage TAD drying process. The temperature of the dryer was held at 110° C. during both drying stages.

### EXAMPLE 2

A three layer patterned non-woven web was provided, with the bottom layer being 100% polypropylene and the top two layers being made of 60/40 polypropylene/polyethylene side-by-side bicomponent fibers. The web had a basis weight of 66.2 gsm and a thickness of 0.556 mm. The patterned non-woven web was produced by first bonding the layers together and subjecting the web to hydroentanglement and then a two-stage TAD drying process. The temperature of the dryer was held at 110° C. during both drying stages.

### EXAMPLE 3

A three layer patterned non-woven web was provided, with the bottom layer being 100% polypropylene and the top two layers being made of 60/40 polypropylene/polyethylene side-by-side bicomponent fibers. The web had a basis weight of 64.4 gsm and a thickness of 0.521 mm. The patterned non-woven web was produced by first bonding the layers together and subjecting the web to hydroentanglement and then a two-stage TAD drying process. The temperature of the dryer was held at 120° C. during both drying stages.

EXAMPLE 4

A three layer patterned non-woven web was provided, with the bottom layer being 100% polypropylene and the top two layers being made of 60/40 polypropylene/polyethylene side-by-side bicomponent fibers. The web had a basis weight of 33.0 gsm and a thickness of 0.374 mm. The patterned non-woven web was produced by first bonding the layers together and subjecting the web to hydroentanglement and then a two-stage TAD drying process. The temperature of the dryer was held at 120° C. during both drying stages.

### EXAMPLE 5

A three layer patterned non-woven web was provided, with the bottom layer being 100% polypropylene and the top two layers being made of 60/40 polypropylene/polyethylene side-by-side bicomponent fibers. The web had a basis weight of 35.3 gsm and a thickness of 0.350 mm. The patterned non-woven web was produced by first bonding the layers together and subjecting the web to hydroentanglement and then a two-stage TAD drying process. The temperature of the dryer was held at 130° C. during both drying stages.

EXAMPLE 6

A three layer patterned non-woven web was provided, with the bottom layer being 100% polypropylene and the top two layers being made of 60/40 polypropylene/polyethylene side-by-side bicomponent fibers. The web had a basis weight of 69.0 gsm and a thickness of 0.520 mm. The patterned non-woven web was produced by first bonding the layers together and subjecting the web to hydroentanglement and then a two-stage TAD drying process. The temperature of the dryer was held at 130° C. during both drying stages.

For each of Examples 1-6, tensile strength in the machine direction and cross direction and elongation in the machine direction were measured. The tensile strength and elongation were measured using the WSP 110.4 B test method. The results are shown in Table 1 below.

TABLE 1

| SAMPLE ID | 1 | 4 | 5 | 2 | 3 | 6 |
|---|---|---|---|---|---|---|
| BASIS WEIGHT (GSM) | 30.9 | 33.0 | 35.3 | 66.2 | 64.4 | 69.0 |
| THICKNESS (MM) | 0.363 | 0.374 | 0.350 | 0.556 | 0.521 | 0.520 |
| MDT (G/CM) | 1286 | 1208 | 1473 | 3368 | 3474 | 3590 |
| MDE (%) | 103 | 79 | 52 | 128 | 99 | 67 |
| CDT (G/CM) | 504 | 476 | 609 | 1144 | 1160 | 1312 |
| DRYER TEMP. (° C.) | 110 | 120 | 130 | 110 | 120 | 130 |

The results shown in Table 1 indicate that there is a significant increase in MD and CD tensile strength as well as a significant reduction in MD elongation for the samples, independent of basis weight, as the dryer temperature approaches the melting temperature of polyethylene. Without being bound by theory, it is believed that the melting of the polyethylene in the top layers of the samples during the second stage of the TAD drying process contributes to the increase in tensile strength and decrease in elongation.

Also, for each of Examples 1-6, an abrasion rating was determined based on a visual rating scale of 1 to 5, with 1 indicating little to no evidence of surface abrasion while 5 suggests significant fiber ‘pilling’ and ‘roping’ evident on the surface following abrasion. As shown in FIG. 3, for both 30 gsm and 60 gsm basis weights, the abrasion rating dropped with increased dryer temperature. Without being bound by theory, this is again contributed to the bonding that takes place within the web as the dryer temperature is increased. The difference observed as to when the improvement in abrasion is noted between the two basis weight samples is considered a function of the material dwell time on the dryer surface.

While particular embodiments of the invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A nonwoven web comprising:
a first layer comprising continuous fibers including a first polymer component; and
a second layer comprising continuous fibers including a second polymer component, the melting point of the second polymer component being less than the melting point of the first polymer component, the first layer being hydroentangled with the second layer.

2. The nonwoven web of claim 1, wherein the first polymer component is polypropylene.

3. The nonwoven web of claim 1, wherein the second polymer component is polyethylene.

4. The nonwoven web of claim 3, wherein the continuous fibers of the second layer further include the first polymer component.

5. The nonwoven web of claim 4, wherein the continuous fibers of the second layer are bicomponent fibers.

6. The nonwoven web of claim 1, wherein the first layer comprises a pattern.

7. The nonwoven web of claim 5, wherein the bicomponent fibers are arranged in a side by side configuration.

8. The nonwoven web of claim 1, wherein the nonwoven web has a basis weight within the range of 30 gsm to 35 gsm and a tensile strength in the machine direction of at least 1400 g/cm.

9. The nonwoven web of claim 8, wherein the nonwoven web has a tensile strength in the cross direction of at least 500 g/cm.

10. The nonwoven web of claim 8, wherein the nonwoven web has a percent elongation in the machine direction within the range of 50 to 100.

11. An absorbent article including from top to bottom a topsheet, an absorbent core, and a backsheet, wherein the topsheet comprises the nonwoven web of claim 1.

12. The nonwoven web of claim 1, wherein the nonwoven web has a basis weight within the range of 60 gsm to 70 gsm and a tensile strength in the machine direction of at least 3400 g/cm.

13. The nonwoven web of claim 12, wherein the nonwoven web has a tensile strength in the cross direction of at least 1100 g/cm.

14. The nonwoven web of claim 12, wherein the nonwoven web has a percent elongation in the machine direction within the range of 60 to 130.

15. A method of making a patterned nonwoven web, comprising:
forming a nonwoven web comprising:
a first layer comprising continuous fibers; and
a second layer comprising continuous fibers, the melting point of the continuous fibers of the second layer being less than the melting point of the continuous fibers of the first layer;
thermal bonding the first layer to the second layer;
subjecting the thermally bonded first and second layers to a hydroentanglement process so as to form a pattern in the first layer; and
through air drying the hydroentangled first and second layers so that the continuous fibers of the second layer melt.

16. The method of claim 15, wherein the step of forming a nonwoven web comprises a spunmelt process.

17. The method of claim 15, wherein the thermal bonding is done at a reduced temperature relative to a standard temperature for thermal bonding.

18. The method of claim 15, wherein the continuous fibers of the first layer are made of polypropylene.

19. The method of claim 15, wherein the continuous fibers of the second layer are made of polyethylene.

20. The method of claim 15, wherein the through-air drying step comprises a two-stage through-air drying process.

21. The method of claim 20, wherein the two-stage through-air drying process comprises a first stage through-air drying process in which the nonwoven web is dried and a second stage through-air drying process in which the continuous fibers of the second layer are melted.

22. An absorbent article including from top to bottom a topsheet, an absorbent core, and a backsheet, wherein the absorbent article further comprises a first layer located between the topsheet and the absorbent core wherein the first layer comprises the nonwoven web of claim 1.
